(19) United States
(12) Patent Application Publication
(10) Pub. No.: US 2026/0275316 A1
(43) Pub. Date: Sep. 17, 2026

(54) MUTANT OF TYPE III PULLULAN HYDROLASE WITH ENHANCED ENZYMATIC ACTIVITY AND CONSTRUCTION METHOD THEREOF

(71) Applicant: Institute of Microbiology, Jiangxi Academy of Sciences (Jiangxi Institute of Watershed Ecology), Nanchang City (CN)

(72) Inventors: Jing ZENG, Nanchang City (CN); Xuaner LUO, Nanchang City (CN); Lin YUAN, Nanchang City (CN); Jianjun GUO, Nanchang City (CN); Dawei XIONG, Nanchang City (CN); Shuaiwen ZHANG, Nanchang City (CN); Tong WANG, Nanchang City (CN)

(21) Appl. No.: 19/358,718

(22) Filed: Oct. 15, 2025

(30) Foreign Application Priority Data
Mar. 11, 2025 (CN) 202510283939.1

(51) Int. Cl. C12N 9/44

(52) U.S. Cl. CPC C12N 9/2457; C12Y 302/01041

(57) ABSTRACT

A mutant of Type III pullulan hydrolase with enhanced enzymatic activity and a construction method thereof are provided. The amino acid sequence of the type III pullulan hydrolase mutant is shown in SEQ ID NO. 3. On the basis of type III pullulan hydrolase TK-PUL from Thermococcus kodakarensis, the disclosure develops a mutant of type III pullulan hydrolase with enhanced enzymatic activity through site-directed mutagenesis, shows a 1.83-fold increase in specific enzymatic activity against cassava starch.

### CROSS-REFERENCE TO RELATED APPLICATIONS

This application claims priority to Chinese Patent Application No. 202510283939.1, filed on Mar. 11, 2025, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Oct. 13, 2025
Byte size: 90,667

### TECHNICAL FIELD

The present application relates to the fields of genetic engineering and enzyme engineering, and in particular to a mutant of Type III pullulan hydrolase with enhanced enzymatic activity and a construction method thereof.

### BACKGROUND

Linear maltooligosaccharides typically refer to linear oligosaccharides composed of 2 to 10 glucose molecules linked by α-1,4-glycosidic bonds. Due to their low sweetness, ability to delay starch aging, and roles in maintaining blood sugar balance and improving intestinal environment, they are widely used in the food industry.

Currently, the industrial production of linear maltooligosaccharides primarily involves enzymatic hydrolysis of starch. The production process includes two steps: liquefaction and saccharification. In these steps, various glycoside hydrolases hydrolyze the α-1,4-glycosidic and α-1,6-glycosidic bonds in starch, ultimately producing linear maltooligosaccharides. The use of multiple glycoside hydrolases complicates the production process of linear maltooligosaccharides and increases costs.

Thermophilic acidic Type III pullulan hydrolase Thermococcus kodakarensis Pullulanase (TK-PUL) from thermophilic archaeon Thermococcus kodakarensis may hydrolyze both α-1,4-glycosidic and α-1,6-glycosidic bonds in starch. Applying TK-PUL in the production of linear maltooligosaccharides may combine the liquefaction and saccharification steps, significantly simplifying the production process, reducing costs, and improving efficiency. Previous studies have shown that adding TK-PUL at a concentration of 1 milligram per gram (mg/g) dry starch to a 30 percent (%) corn starch slurry (100 millimolar (mM) sodium citrate buffer, pH 4.2), reacting at 100 degrees Celsius (° C.) for 10 minutes (min), and then at 90° C. for 96 hours (h), results in a product where G2-G10 constitutes 85.5% of the total. This indicates that TK-PUL has significant potential in the production of linear maltooligosaccharides. However, the catalytic efficiency of TK-PUL is relatively low, and it is difficult to effectively play the role of hydrolysis in actual industrial production, which limits the application of TK-PUL in the production and preparation of linear maltooligosaccharides. Additionally, compared to traditional raw materials like corn and wheat starch, non-grain raw materials such as cassava starch are more widely available and cost-effective, making them promising alternatives for biomass energy, and having great application and development prospect. There is currently no research on the use of Type III pullulan hydrolase with cassava starch as a raw material for producing linear maltooligosaccharides. Therefore, developing a mutant of Type III pullulan hydrolase with enhanced cassava starch degradation capability is of great importance for its application in producing linear maltooligosaccharides from cassava starch.

### SUMMARY

The objectives of the present disclosure are to provide a mutant of Type III pullulan hydrolase with enhanced enzymatic activity and its construction method, addressing the issues in the existing technology. The mutant of Type III pullulan hydrolase prepared by this disclosure has high cassava starch degradation capability, and its enzymatic properties meet the requirements for the production process of linear maltooligosaccharides, indicating significant application potential in the production of linear maltooligosaccharides.

To achieve the objectives, the present disclosure provides the following schemes.

The present disclosure provides a mutant of Type III pullulan hydrolase, where the amino acid sequence is shown in SEQ ID NO. 3.

The present disclosure also provides a coding gene encoding the mutant of Type III pullulan hydrolase.

In an embodiment, the nucleotide sequence of the encoding gene is shown in SEQ ID NO. 4.

The present disclosure also provides a recombinant vector, including the encoding gene.

The present disclosure also provides a recombinant microbial strain, including the recombinant vector.

The present disclosure also provides an application of the encoding gene, the recombinant vector or the recombinant microbial strain in the preparation of the mutant of Type III pullulan hydrolase.

The present disclosure also provides an application of the mutant of Type III pullulan hydrolase in the production of linear maltooligosaccharides using cassava starch as a raw material.

The present disclosure also provides a preparation method of the linear maltooligosaccharide, which includes the step of preparing the linear maltooligosaccharide by using cassava starch as a substrate and the type II pullulan hydrolase mutant as an enzyme catalyst through an enzymatic hydrolysis reaction.

In an embodiment, the mass ratio of the type III pullulan hydrolase mutant to the cassava starch is (1-4) milligram (mg): 1 gram (g).

The present disclosure also provides a method for enhancing the enzymatic activity of Type III pullulan hydrolase TK-PUL in catalyzing the hydrolysis of cassava starch to produce linear maltooligosaccharides, where the amino acid sequence of Type III pullulan hydrolase TK-PUL is shown in SEQ ID NO. 1.

The method includes the steps of mutating the 282nd amino acid to arginine (R) and the 283rd amino acid to glutamic acid (E).

The present disclosure discloses the following technical effects.

The disclosure provides a type III pullulan hydrolase mutant with enhanced enzymatic activity. The type III pullulan hydrolase mutant is based on the type III pullulan hydrolase TK-PUL derived from Thermococcus kodakarensis, and its enzymatic activity is improved through site-directed mutagenesis. The specific enzymatic activity of this type III pullulan hydrolase mutant on cassava starch increases from 48.68 Unit per milligram (U/mg) (before mutation) to 89.08 U/mg, representing a 1.83-fold improvement. Using cassava starch as the substrate, the optimal reaction temperature for the type II pullulan hydrolase mutant is 100 degrees Celsius (° C.), and the optimal reaction pH is 4.5, its half-life at 90° C. is 20 hours (h). When 30 percent (%) cassava starch is used as the substrate and 4 milligrams per gram (mg/g) dry cassava starch of the type III pullulan hydrolase is added, the substrate conversion rate of the linear maltooligosaccharides G2-G10 in the product increases from 84.50% (before mutation) to 92.23%. The cassava starch degradation capability of this type III pullulan hydrolase mutant is significantly enhanced, and its enzymatic properties meet the requirements for the production and preparation of linear maltooligosaccharides, thereby improving its application potential in the production and preparation of linear maltooligosaccharides.

### BRIEF DESCRIPTION OF THE DRAWINGS

To provide a clearer explanation of the technical schemes in the embodiments of the present disclosure or the prior art, the following will briefly introduce the accompanying drawings required for the embodiments. Obviously, the drawings described below are only some embodiments of the present disclosure. For those of ordinary skill in the art, other drawings may be obtained based on these illustrations without the need for creative effort.

FIG. 1 shows the statistical diagram of relative enzymatic activity of Type III pullulan hydrolase Thermococcus kodakarensis Pullulanase (TK-PUL) and mutant C282R/C283E at different reaction temperatures.

FIG. 2 shows the statistical diagram of relative enzymatic activity of Type III pullulan hydrolase TK-PUL and mutant C282R/C283E at different reaction pH levels.

FIG. 3 shows the statistical diagram of thermal stability of Type III pullulan hydrolase TK-PUL and mutant C282R/C283E at 90 degrees Celsius (° C.).

FIG. 4 shows the statistical diagram of substrate conversion rate of Type III pullulan hydrolase TK-PUL and mutant C282R/C283E for preparing linear maltooligosaccharides (G2-G10) with cassava starch as a substrate.

### DETAILED DESCRIPTION OF THE EMBODIMENTS

The present disclosure is now described in detail with reference to various exemplary embodiments. This detailed description should not be construed as limiting the disclosure but rather as providing a more detailed explanation of certain aspects, features, and implementations of the disclosure.

It should be understood that the terms used in the present disclosure are only for describing specific embodiments and are not intended to limit the disclosure. Additionally, for numerical ranges described in the disclosure, it should be understood that each intermediate value between the upper and lower limits of the range is also specifically disclosed. Any stated value or intermediate value within a stated range, as well as any smaller range formed by any other stated value or intermediate value within the stated range, is also included in the disclosure. The upper and lower limits of these smaller ranges may be independently included or excluded from the range.

Without departing from the scope or spirit of the disclosure, various modifications and changes may be made to the specific embodiments described in the specification, which will be obvious to those skilled in the art. Other embodiments derived from the specification of the disclosure will also be obvious to those skilled in the art. The specification and embodiments of the disclosure are merely illustrative.

Regarding the terms “comprising,” “including,” “having,” “containing,” etc., used herein, they are open-ended terms, meaning including but not limited to.

The experimental materials used in the following embodiments are as follows.

### 1. Strains and Vectors

Escherichia coli JM109 (provided by Jiangxi Academy of Sciences Institute of Microbiology), Bacillus subtilis WB600 (provided by Jiangxi Academy of Sciences Institute of Microbiology), and the Bacillus subtilis expression vector pSTOP1622 (purchased from MoBiTec GmbH).

### 2. Enzymes and Other Biochemical Reagents

The site-directed mutagenesis kit was purchased from Beyotime Biotechnology Co., Ltd., Shanghai. KOD-Plus-neo DNA polymerase was purchased from Toyobo Co., Ltd. DNA restriction enzymes and T4 DNA ligase were purchased from Fermentas Company, DNA gel recovery kit and plasmid extraction kit E.Z.N.A. were purchased from Omega Bio-tek Company, Chelating Sepharose™ Fast Flow was purchased from GE Healthcare, USA, cassava starch was purchased from Beijing Bio-Leader Technology Co., Ltd, and other chemical reagents were all of domestic or imported analytical grade.

### 3. Culture Media

Luria-Bertani (LB medium): 10 grams per liter (g/L) of tryptone, 5 g/L of yeast extract, 10 g/L of NaCl, pH 7.0. The screening medium is LB medium containing 100 micrograms per milliliter (μg/mL) of ampicillin.

The molecular cloning techniques and protein detection techniques used in the disclosure are conventional techniques in the field. Techniques not described in detail in the following embodiments are carried out according to the relevant sections of the following experimental manual: Green M R, Sambrook J. Molecular Cloning: A Laboratory Manual [M]. New York: Cold Spring Harbor Laboratory Press, 2012.

### 4. Wild-Type Enzyme and its Mutants

The type III pullulan hydrolase mutants of the disclosure are obtained by site-directed mutagenesis based on the type III pullulan hydrolase Thermococcus kodakarensis Pullulanase (TK-PUL) derived from Thermococcus kodakarensis, where the amino acid sequence of type III pullulan hydrolase TK-PUL is shown in SEQ ID NO. 1, and the nucleotide sequence of its encoding gene is shown in SEQ ID NO. 2; the amino acid sequence of the type III pullulan hydrolase mutant C282R/C283E is shown in SEQ ID NO. 3, and the nucleotide sequence of its encoding gene is shown in SEQ ID NO. 4.

SEQ ID NO. 1:
MKKGGLLLILLILVSIAEKGCPSGRVPVKFTYNPGNKTVKSVSLRGSFNNWGE
WPMELKNGTWETTVCLRPGRYEYKYFINGQWVKDMSDDGTGRPYDPDADAYAPDGY
GGKNAVRVVEGREAFYVEFDPRDPAYLSIADKRTVVRFEAKRDTVESAVLVTDHGNYT
MKLQVWWDFGETWRAEMPVEPADYYILVTSSDGGKFAVLNTSESPFFHFDGVEGFPQL
EWVSNGITYQIFPDRFNNGNKSNDALALDHDELILNQVNPGQPILSNWSDPITPLHCCHQ
YFGGDIKGITEKLDYLQSLGVTIIYINPIFLSGSAHGYDTYDYYRLDPKFGTEDELREFLD
EAHRRGMRVIFDFVPNHCGIGNPAFLDVWEKGNESPYWDWFFVKKWPFKLGDGSAYV
GWWGFGSLPKLNTANQEVREYLIGAALHWIEFGFDGIRVDVPNEVLDPGTFFPELRKAV
KEKNPDAYLVGEIWTESPEWVKGDRFDSLMNYALGRDILLNYAKGLLSGESAMKMMG
RYYASYGENVVAMGFNLVDSHDTSRVLTDLGGGKLGDTPSNESIQRLKLLSTLLYALPG
TPVTFQGDERGLLGDKGHYDEQRYPIQWDTVNEDVLNHYRALAELRKRVPALRSSAM
RFYTAKGGVMAFFRGHHDEVLVVANSWKKPALLELPEGEWKVIWPEDFSPELLRGTVE
VPAIGIIILERGHHHHHH.
SEQ ID NO. 2:
ATGAAAAAAGGTGGTCTGCTGCTCATTCTCCTGATTCTGGTCTCAATCGCC
GAGAAAGGCTGTCCCTCCGGAAGAGTCCCGGTGAAGTTCACGTACAACCCCGGAAA
CAAGACCGTAAAGTCTGTCAGCCTCCGCGGGAGCTTCAACAACTGGGGAGAGTGGC
CGATGGAGCTGAAGAACGGCACGTGGGAGACGACCGTCTGTCTCCGCCCTGGAAGG
TATGAGTATAAGTACTTCATCAACGGCCAGTGGGTCAAGGACATGTCCGACGACGG
GACGGGAAGGCCCTACGACCCCGATGCAGACGCCTATGCCCCCGATGGCTACGGGG
GAAAGAACGCCGTGAGGGTAGTTGAGGGCCGCGAAGCGTTCTACGTGGAGTTCGAT
CCAAGAGACCCAGCCTACCTCAGCATCGCGGACAAAAGAACCGTGGTCAGGTTCGA
GGCTAAGAGAGACACCGTCGAGTCTGCGGTTCTCGTTACGGATCACGGGAACTACA
CGATGAAGCTTCAGGTCTGGTGGGACTTCGGCGAAACCTGGCGCGCCGAGATGCCA
GTTGAACCCGCTGATTATTACATTCTCGTAACCTCCTCCGACGGCGGGAAGTTTGCC
GTCCTAAACACAAGCGAAAGCCCGTTCTTCCACTTTGATGGCGTTGAGGGGTTCCCC
CAGCTGGAGTGGGTGAGCAACGGGATAACCTACCAGATATTCCCCGACAGGTTCAA
CAACGGCAATAAAAGCAACGATGCCCTAGCTTTGGATCACGACGAGCTAATTTTGA
ACCAGGTTAATCCAGGGCAGCCAATCCTCTCCAACTGGAGCGACCCGATAACGCCC
CTCCACTGCTGCCACCAGTACTTCGGCGGCGACATAAAGGGAATAACGGAGAAGCT
CGACTACCTTCAGAGCCTAGGTGTTACTATAATCTACATCAACCCGATTTTCCTCTCG
GGAAGCGCCCACGGCTACGACACCTACGACTACTACCGGCTCGACCCCAAGTTCGG
GACCGAGGATGAGCTGAGAGAGTTCCTCGATGAGGCCCACAGGAGGGGAATGAGG
GTAATCTTCGATTTCGTGCCCAACCACTGCGGCATAGGGAATCCAGCCTTCCTCGAC
GTCTGGGAGAAGGGCAACGAAAGCCCATACTGGGACTGGTTCTTCGTCAAGAAGTG
GCCCTTCAAGCTCGGCGATGGGAGCGCCTACGTCGGCTGGTGGGGCTTTGGGAGCCT
TCCGAAGCTCAACACTGCCAACCAGGAGGTCAGGGAGTACCTGATAGGAGCGGCCC
TCCACTGGATAGAGTTCGGCTTTGACGGCATTAGGGTGGATGTGCCGAACGAAGTCC
TCGACCCGGGGACGTTCTTCCCGGAGCTGAGAAAGGCAGTTAAGGAGAAAAACCCC
GACGCGTACCTCGTCGGCGAGATATGGACGGAATCCCCGGAGTGGGTGAAGGGAGA
CCGCTTCGACTCCCTCATGAACTACGCCCTCGGGAGGGACATCCTCCTGAACTACGC
TAAGGGCCTGCTCAGCGGAGAAAGTGCAATGAAAATGATGGGACGTTACTACGCTT
CCTACGGCGAGAACGTAGTTGCGATGGGCTTCAACCTCGTTGATTCGCACGACACTT
CGAGGGTTCTCACTGACCTCGGTGGTGGCAAACTGGGAGACACACCGTCAAACGAG
TCAATTCAGAGGCTCAAGCTCCTCTCAACGCTCCTCTATGCCCTGCCCGGAACTCCC
GTCACCTTCCAGGGGGACGAGAGGGGACTGCTCGGAGACAAGGGACACTACGATGA
GCAACGCTATCCGATACAGTGGGATACTGTGAACGAGGACGTCCTGAACCACTACA
GGGCACTGGCGGAGCTCAGAAAAAGAGTTCCCGCATTGAGGAGCAGCGCAATGAG
GTTCTACACTGCCAAAGGCGGCGTTATGGCCTTCTTCAGGGGACATCATGACGAGGT
TCTCGTCGTTGCCAACAGCTGGAAGAAGCCAGCCCTACTGGAGCTTCCCGAGGGAG
AGTGGAAAGTAATCTGGCCTGAGGATTTCAGCCCGGAACTGCTTCGCGGCACAGTT
GAAGTGCCAGCCATAGGGATAATCATCCTTGAGCGGGGTCATCATCATCATCATCAT
TGA.
SEQ ID NO. 3:
MKKGGLLLILLILVSIAEKGCPSGRVPVKFTYNPGNKTVKSVSLRGSFNNWGE
WPMELKNGTWETTVCLRPGRYEYKYFINGQWVKDMSDDGTGRPYDPDADAYAPDGY
GGKNAVRVVEGREAFYVEFDPRDPAYLSIADKRTVVRFEAKRDTVESAVLVTDHGNYT
MKLQVWWDFGETWRAEMPVEPADYYILVTSSDGGKFAVLNTSESPFFHFDGVEGFPQL
EWVSNGITYQIFPDRFNNGNKSNDALALDHDELILNQVNPGQPILSNWSDPITPLHREHQ
YFGGDIKGITEKLDYLQSLGVTIIYINPIFLSGSAHGYDTYDYYRLDPKFGTEDELREFLD
EAHRRGMRVIFDFVPNHCGIGNPAFLDVWEKGNESPYWDWFFVKKWPFKLGDGSAYV
GWWGFGSLPKLNTANQEVREYLIGAALHWIEFGFDGIRVDVPNEVLDPGTFFPELRKAV
KEKNPDAYLVGEIWTESPEWVKGDRFDSLMNYALGRDILLNYAKGLLSGESAMKMMG
RYYASYGENVVAMGFNLVDSHDTSRVLTDLGGGKLGDTPSNESIQRLKLLSTLLYALPG
TPVTFQGDERGLLGDKGHYDEQRYPIQWDTVNEDVLNHYRALAELRKRVPALRSSAM
RFYTAKGGVMAFFRGHHDEVLVVANSWKKPALLELPEGEWKVIWPEDFSPELLRGTVE
VPAIGIIILERGHHHHHH.
SEQ ID NO. 4:
ATGAAAAAAGGTGGTCTGCTGCTCATTCTCCTGATTCTGGTCTCAATCGCC
GAGAAAGGCTGTCCCTCCGGAAGAGTCCCGGTGAAGTTCACGTACAACCCCGGAAA
CAAGACCGTAAAGTCTGTCAGCCTCCGCGGGAGCTTCAACAACTGGGGAGAGTGGC
CGATGGAGCTGAAGAACGGCACGTGGGAGACGACCGTCTGTCTCCGCCCTGGAAGG
TATGAGTATAAGTACTTCATCAACGGCCAGTGGGTCAAGGACATGTCCGACGACGG
GACGGGAAGGCCCTACGACCCCGATGCAGACGCCTATGCCCCCGATGGCTACGGGG
GAAAGAACGCCGTGAGGGTAGTTGAGGGCCGCGAAGCGTTCTACGTGGAGTTCGAT
CCAAGAGACCCAGCCTACCTCAGCATCGCGGACAAAAGAACCGTGGTCAGGTTCGA
GGCTAAGAGAGACACCGTCGAGTCTGCGGTTCTCGTTACGGATCACGGGAACTACA
CGATGAAGCTTCAGGTCTGGTGGGACTTCGGCGAAACCTGGCGCGCCGAGATGCCA
GTTGAACCCGCTGATTATTACATTCTCGTAACCTCCTCCGACGGCGGGAAGTTTGCC
GTCCTAAACACAAGCGAAAGCCCGTTCTTCCACTTTGATGGCGTTGAGGGGTTCCCC
CAGCTGGAGTGGGTGAGCAACGGGATAACCTACCAGATATTCCCCGACAGGTTCAA
CAACGGCAATAAAAGCAACGATGCCCTAGCTTTGGATCACGACGAGCTAATTTTGA
ACCAGGTTAATCCAGGGCAGCCAATCCTCTCCAACTGGAGCGACCCGATAACGCCC
CTCCACCGCGAACACCAGTACTTCGGCGGCGACATAAAGGGAATAACGGAGAAGCT
CGACTACCTTCAGAGCCTAGGTGTTACTATAATCTACATCAACCCGATTTTCCTCTCG
GGAAGCGCCCACGGCTACGACACCTACGACTACTACCGGCTCGACCCCAAGTTCGG
GACCGAGGATGAGCTGAGAGAGTTCCTCGATGAGGCCCACAGGAGGGGAATGAGG
GTAATCTTCGATTTCGTGCCCAACCACTGCGGCATAGGGAATCCAGCCTTCCTCGAC
GTCTGGGAGAAGGGCAACGAAAGCCCATACTGGGACTGGTTCTTCGTCAAGAAGTG
GCCCTTCAAGCTCGGCGATGGGAGCGCCTACGTCGGCTGGTGGGGCTTTGGGAGCCT
TCCGAAGCTCAACACTGCCAACCAGGAGGTCAGGGAGTACCTGATAGGAGCGGCCC
TCCACTGGATAGAGTTCGGCTTTGACGGCATTAGGGTGGATGTGCCGAACGAAGTCC
TCGACCCGGGGACGTTCTTCCCGGAGCTGAGAAAGGCAGTTAAGGAGAAAAACCCC
GACGCGTACCTCGTCGGCGAGATATGGACGGAATCCCCGGAGTGGGTGAAGGGAGA
CCGCTTCGACTCCCTCATGAACTACGCCCTCGGGAGGGACATCCTCCTGAACTACGC
TAAGGGCCTGCTCAGCGGAGAAAGTGCAATGAAAATGATGGGACGTTACTACGCTT
CCTACGGCGAGAACGTAGTTGCGATGGGCTTCAACCTCGTTGATTCGCACGACACTT
CGAGGGTTCTCACTGACCTCGGTGGTGGCAAACTGGGAGACACACCGTCAAACGAG
TCAATTCAGAGGCTCAAGCTCCTCTCAACGCTCCTCTATGCCCTGCCCGGAACTCCC
GTCACCTTCCAGGGGGACGAGAGGGGACTGCTCGGAGACAAGGGACACTACGATGA
GCAACGCTATCCGATACAGTGGGATACTGTGAACGAGGACGTCCTGAACCACTACA
GGGCACTGGCGGAGCTCAGAAAAAGAGTTCCCGCATTGAGGAGCAGCGCAATGAG
GTTCTACACTGCCAAAGGCGGCGTTATGGCCTTCTTCAGGGGACATCATGACGAGGT
TCTCGTCGTTGCCAACAGCTGGAAGAAGCCAGCCCTACTGGAGCTTCCCGAGGGAG
AGTGGAAAGTAATCTGGCCTGAGGATTTCAGCCCGGAACTGCTTCGCGGCACAGTT
GAAGTGCCAGCCATAGGGATAATCATCCTTGAGCGGGGTCATCATCATCATCATCAT
TGA.

### Embodiment 1: Construction of Type III Pullulan Hydrolase Mutants

(1) Construction of the Recombinant Plasmid pSTOP1622-Tkpul

Using the nucleotide sequence shown in SEQ ID NO. 2 as a template and primers P1 and P2 (Table 1), PCR amplification is performed. The PCR conditions are as follows: 95 degrees Celsius (° C.) for 10 minutes (min); 98° C. for 30 seconds (s), 60° C. for 30 s, 74° C. for 1 min, 30 cycles; and 74° C. for 5 min. The amplified product is double-digested with Spe I and BamH I and ligated into the vector pSTOP1622 to construct the recombinant plasmid pSTOP1622-tkpul.

TABLE 1
primers used for constructing the recombinant plasmid

| Primer | | Sequence |
|---|---|---|
| Name | Nucleotide Sequence | Number |
| P1 | 5′-AGTACTAGTATGAAAAAAGGTGGTCTGCTGCTCATTCTC- | SEQ ID NO. 5 |
| | 3′ | |
| P2 | 5′-TGGGATCCTCAATGATGATGATGATGATGAC-3′ | SEQ ID NO. 6 |
| C282A-F | 5′-GATAACGCCCCTCCACGCCTGCCACCAGTACTTCG-3′ | SEQ ID NO. 7 |
| C282A-R | 5′-CGAAGTACTGGTGGCAGGCGTGGAGGGGCGTTATC-3′ | SEQ ID NO. 8 |
| C282G-F | 5′-GATAACGCCCCTCCACGGCTGCCACCAGTACTTCG-3′ | SEQ ID NO. 9 |
| C282G-R | 5′-CGAAGTACTGGTGGCAGCCGTGGAGGGGCGTTATC-3′ | SEQ ID NO. 10 |
| C282V-F | 5′-GATAACGCCCCTCCACGTCTGCCACCAGTACTTCG-3′ | SEQ ID NO. 11 |
| C282V-R | 5′-CGAAGTACTGGTGGCAGACGTGGAGGGGCGTTATC-3′ | SEQ ID NO. 12 |
| C282L-F | 5′-GATAACGCCCCTCCACCTCTGCCACCAGTACTTCG-3′ | SEQ ID NO. 13 |
| C282L-R | 5′-CGAAGTACTGGTGGCAGAGGTGGAGGGGCGTTATC-3′ | SEQ ID NO. 14 |
| C282I-F | 5′-GATAACGCCCCTCCACATCTGCCACCAGTACTTCG-3′ | SEQ ID NO. 15 |
| C282I-R | 5′-CGAAGTACTGGTGGCAGATGTGGAGGGGCGTTATC-3′ | SEQ ID NO. 16 |
| C282P-F | 5′-GATAACGCCCCTCCACCCCTGCCACCAGTACTTCG-3′ | SEQ ID NO. 17 |
| C282P-R | 5′-CGAAGTACTGGTGGCAGGGGTGGAGGGGCGTTATC-3′ | SEQ ID NO. 18 |
| C282F-F | 5′-GATAACGCCCCTCCACTTCTGCCACCAGTACTTCG-3′ | SEQ ID NO. 19 |
| C282F-R | 5′-CGAAGTACTGGTGGCAGAAGTGGAGGGGCGTTATC-3′ | SEQ ID NO. 20 |
| C282W-F | 5′-GATAACGCCCCTCCACTGGTGCCACCAGTACTTCG-3′ | SEQ ID NO. 21 |
| C282W-R | 5′-CGAAGTACTGGTGGCACCAGTGGAGGGGCGTTATC-3′ | SEQ ID NO. 22 |
| C282D-F | 5′-GATAACGCCCCTCCACGACTGCCACCAGTACTTCG-3′ | SEQ ID NO. 23 |
| C282D-R | 5′-CGAAGTACTGGTGGCAGTCGTGGAGGGGCGTTATC-3′ | SEQ ID NO. 24 |
| C282E-F | 5′-GATAACGCCCCTCCACGAATGCCACCAGTACTTCG-3′ | SEQ ID NO. 25 |
| C282E-R | 5′-CGAAGTACTGGTGGCATTCGTGGAGGGGCGTTATC-3′ | SEQ ID NO. 26 |
| C282T-F | 5′-GATAACGCCCCTCCACACCTGCCACCAGTACTTCG-3′ | SEQ ID NO. 27 |
| C282T-R | 5′-CGAAGTACTGGTGGCAGGTGTGGAGGGGCGTTATC-3′ | SEQ ID NO. 28 |
| C282N-F | 5′-GATAACGCCCCTCCACAACTGCCACCAGTACTTCG-3′ | SEQ ID NO. 29 |
| C282N-R | 5′-CGAAGTACTGGTGGCAGTTGTGGAGGGGCGTTATC-3′ | SEQ ID NO. 30 |
| C282K-F | 5′-GATAACGCCCCTCCACAAATGCCACCAGTACTTCG-3′ | SEQ ID NO. 31 |
| C282K-R | 5′-CGAAGTACTGGTGGCATTTGTGGAGGGGCGTTATC-3′ | SEQ ID NO. 32 |
| C282S-F | 5′-GATAACGCCCCTCCACAGCTGCCACCAGTACTTCG-3′ | SEQ ID NO. 33 |
| C282S-R | 5′-CGAAGTACTGGTGGCAGCTGTGGAGGGGCGTTATC-3′ | SEQ ID NO. 34 |
| C282R-F | 5′-GATAACGCCCCTCCACAGATGCCACCAGTACTTCG-3′ | SEQ ID NO. 35 |
| C282R-R | 5′-CGAAGTACTGGTGGCATCTGTGGAGGGGCGTTATC-3′ | SEQ ID NO. 36 |
| C282H-F | 5′-GATAACGCCCCTCCACCACTGCCACCAGTACTTCG-3′ | SEQ ID NO. 37 |
| C282H-R | 5′-CGAAGTACTGGTGGCAGTGGTGGAGGGGCGTTATC-3′ | SEQ ID NO. 38 |
| C282Q-F | 5′-GATAACGCCCCTCCACCAATGCCACCAGTACTTCG-3′ | SEQ ID NO. 39 |
| C282Q-R | 5′-CGAAGTACTGGTGGCATTGGTGGAGGGGCGTTATC-3′ | SEQ ID NO. 40 |
| C282Y-F | 5′-GATAACGCCCCTCCACTACTGCCACCAGTACTTCG-3′ | SEQ ID NO. 41 |
| C282Y-R | 5′-CGAAGTACTGGTGGCAGTAGTGGAGGGGCGTTATC-3′ | SEQ ID NO. 42 |
| C282M-F | 5′-GATAACGCCCCTCCACATGTGCCACCAGTACTTCG-3′ | SEQ ID NO. 43 |
| C282M-R | 5′-CGAAGTACTGGTGGCACATGTGGAGGGGCGTTATC-3′ | SEQ ID NO. 44 |
| C283A-F | 5′-GATAACGCCCCTCCACTGCGCCCACCAGTACTTCG-3′ | SEQ ID NO. 45 |
| C283A-R | 5′-CGAAGTACTGGTGGGCGCAGTGGAGGGGCGTTATC-3′ | SEQ ID NO. 46 |
| C283G-F | 5′-GATAACGCCCCTCCACTGCGGCCACCAGTACTTCG-3′ | SEQ ID NO. 47 |
| C283G-R | 5′-CGAAGTACTGGTGGCCGCAGTGGAGGGGCGTTATC-3′ | SEQ ID NO. 48 |
| C283V-F | 5′-GATAACGCCCCTCCACTGCGTCCACCAGTACTTCG-3′ | SEQ ID NO. 49 |
| C283V-R | 5′-CGAAGTACTGGTGGACGCAGTGGAGGGGCGTTATC-3′ | SEQ ID NO. 50 |
| C283L-F | 5′-GATAACGCCCCTCCACTGCCTCCACCAGTACTTCG-3′ | SEQ ID NO. 51 |
| C283L-R | 5′-CGAAGTACTGGTGGAGGCAGTGGAGGGGCGTTATC-3′ | SEQ ID NO. 52 |
| C283I-F | 5′-GATAACGCCCCTCCACTGCATCCACCAGTACTTCG-3′ | SEQ ID NO. 53 |
| C283I-R | 5′-CGAAGTACTGGTGGATGCAGTGGAGGGGCGTTATC-3′ | SEQ ID NO. 54 |
| C283P-F | 5′-GATAACGCCCCTCCACTGCCCCCACCAGTACTTCG-3′ | SEQ ID NO. 55 |
| C283P-R | 5′-CGAAGTACTGGTGGGGGCAGTGGAGGGGCGTTATC-3′ | SEQ ID NO. 56 |
| C283F-F | 5′-GATAACGCCCCTCCACTGCTTCCACCAGTACTTCG-3′ | SEQ ID NO. 57 |
| C283F-R | 5′-CGAAGTACTGGTGGAAGCAGTGGAGGGGCGTTATC-3′ | SEQ ID NO. 58 |
| C283W-F | 5′-GATAACGCCCCTCCACTGCTGGCACCAGTACTTCG-3′ | SEQ ID NO. 59 |
| C283W-R | 5′-CGAAGTACTGGTGCCAGCAGTGGAGGGGCGTTATC-3′ | SEQ ID NO. 60 |
| C283D-F | 5′-GATAACGCCCCTCCACTGCGACCACCAGTACTTCG-3′ | SEQ ID NO. 61 |
| C283D-R | 5′-CGAAGTACTGGTGGTCGCAGTGGAGGGGCGTTATC-3′ | SEQ ID NO. 62 |
| C283E-F | 5′-GATAACGCCCCTCCACTGCGAACACCAGTACTTCG-3′ | SEQ ID NO. 63 |
| C283E-R | 5′-CGAAGTACTGGTGTTCGCAGTGGAGGGGCGTTATC-3′ | SEQ ID NO. 64 |
| C283T-F | 5′-GATAACGCCCCTCCACTGCACCCACCAGTACTTCG-3′ | SEQ ID NO. 65 |
| C283T-R | 5′-CGAAGTACTGGTGGGTGCAGTGGAGGGGCGTTATC-3′ | SEQ ID NO. 66 |
| C283N-F | 5′-GATAACGCCCCTCCACTGCAACCACCAGTACTTCG-3′ | SEQ ID NO. 67 |
| C283N-R | 5′-CGAAGTACTGGTGGTTGCAGTGGAGGGGCGTTATC-3′ | SEQ ID NO. 68 |
| C283K-F | 5′-GATAACGCCCCTCCACTGCAAACACCAGTACTTCG-3′ | SEQ ID NO. 69 |
| C283K-R | 5′-CGAAGTACTGGTGTTTGCAGTGGAGGGGCGTTATC-3′ | SEQ ID NO. 70 |
| C283S-F | 5′-GATAACGCCCCTCCACTGCAGCCACCAGTACTTCG-3′ | SEQ ID NO. 71 |
| C283S-R | 5′-CGAAGTACTGGTGGCTGCAGTGGAGGGGCGTTATC-3′ | SEQ ID NO. 72 |
| C283R-F | 5′-GATAACGCCCCTCCACTGCAGACACCAGTACTTCG-3′ | SEQ ID NO. 73 |
| C283R-R | 5′-CGAAGTACTGGTGTCTGCAGTGGAGGGGCGTTATC-3′ | SEQ ID NO. 74 |
| C283H-F | 5′-GATAACGCCCCTCCACTGCCACCACCAGTACTTCG-3′ | SEQ ID NO. 75 |
| C283H-R | 5′-CGAAGTACTGGTGGTGGCAGTGGAGGGGCGTTATC-3′ | SEQ ID NO. 76 |
| C283Q-F | 5′-GATAACGCCCCTCCACTGCCAACACCAGTACTTCG-3′ | SEQ ID NO. 77 |
| C283Q-R | 5′-CGAAGTACTGGTGTTGGCAGTGGAGGGGCGTTATC-3′ | SEQ ID NO. 78 |
| C283Y-F | 5′-GATAACGCCCCTCCACTGCTACCACCAGTACTTCG-3′ | SEQ ID NO. 79 |
| C283Y-R | 5′-CGAAGTACTGGTGGTAGCAGTGGAGGGGCGTTATC-3′ | SEQ ID NO. 80 |
| C283M-F | 5′-GATAACGCCCCTCCACTGCATGCACCAGTACTTCG-3′ | SEQ ID NO. 81 |
| C283M-R | 5′-CGAAGTACTGGTGCATGCAGTGGAGGGGCGTTATC-3′ | SEQ ID NO. 82 |
| C282K/ | 5′-GATAACGCCCCTCCACAAAGACCACCAGTACTTCG-3′ | SEQ ID NO. 83 |
| C283D-F | | |
| C282K/ | 5′-CGAAGTACTGGTGGTCTTTGTGGAGGGGCGTTATC-3′ | SEQ ID NO. 84 |
| C283D-R | | |
| C282K/ | 5′-GATAACGCCCCTCCACAAAGAACACCAGTACTTCG-3′ | SEQ ID NO. 85 |
| C283E-F | | |
| C282K/ | 5′-CGAAGTACTGGTGTTCTTTGTGGAGGGGCGTTATC-3′ | SEQ ID NO. 86 |
| C283E-R | | |
| C282K/ | 5′-GATAACGCCCCTCCACAAAAGCCACCAGTACTTCG-3′ | SEQ ID NO. 87 |
| C283S-F | | |
| C282K/ | 5′-CGAAGTACTGGTGGCTTTTGTGGAGGGGCGTTATC-3′ | SEQ ID NO. 88 |
| C283S-R | | |
| C282R/ | 5′-GATAACGCCCCTCCACAGAGACCACCAGTACTTCG-3′ | SEQ ID NO. 89 |
| C283D-F | | |
| C282R/ | 5′-CGAAGTACTGGTGGTCTCTGTGGAGGGGCGTTATC-3′ | SEQ ID NO. 90 |
| C283D-R | | |
| C282R/ | 5′-GATAACGCCCCTCCACAGAGAACACCAGTACTTCG-3′ | SEQ ID NO. 91 |
| C283E-F | | |
| C282R/ | 5′-CGAAGTACTGGTGTTCTCTGTGGAGGGGCGTTATC-3′ | SEQ ID NO. 92 |
| C283E-R | | |
| C282R/ | 5′-GATAACGCCCCTCCACAGAAGCCACCAGTACTTCG-3′ | SEQ ID NO. 93 |
| C283S-F | | |
| C282R/ | 5′-CGAAGTACTGGTGGCTTCTGTGGAGGGGCGTTATC-3′ | SEQ ID NO. 94 |
| C283S-R | | |

Note:
the underlined parts indicate restriction enzyme cutting sites.

### (2) Construction and Screening of Saturation Mutants

The 282nd or 283rd cysteine residues of type III pullulan hydrolase TK-PUL are selected as saturation mutation sites, and 38 mutants are constructed. The specific enzyme activity of the mutants is measured using 1 percent (%) (mass per volume (m/v)) of cassava starch as the substrate.

Construction of mutants: taking the construction of mutant C282A as an example, using the recombinant plasmid pSTOP1622-tkpul as a template and primers C282A-F and C282A-R from Table 1, the site-directed mutagenesis kit is used to mutate the coding sequence of the 282nd cysteine to alanine. The PCR amplification conditions are as follows: 94° C. for 5 min; 94° C. for 30 s, 55° C. for 20 s, 68° C. for 4 min, 35 cycles; and 68° C. for 10 min.

The amplified product is treated with Dpn I enzyme and then electroporated into E. coli JM109 competent cells, coated with LB plate containing 100 μg/mL of ampicillin and cultured overnight at 37° C. Single transformants are picked from the LB plate, and the recombinant plasmid pSTOP1622-tkpulC282A is extracted. Using the recombinant plasmid pSTOP1622-tkpulC282A as a template, PCR amplification is performed with primers P1 and P2, and the amplified DNA is sent to Sangon Biotech Co., Ltd. for sequencing to confirm the presence of the tkpulC282A mutation. The correctly sequenced recombinant plasmid pSTOP1622-tkpul C282A is transformed into B. subtilis WB600 to obtain the engineered strain B. subtilis WB600/pSTOP1622-tkpulC282A.

The construction of other mutants follows the same method as for mutant C282A.

Induced expression and purification of mutants: the engineered strain is inoculated into 20 milliliters (mL) of LB liquid medium containing 20 μg/mL of tetracycline and cultured overnight at 37° C. with vigorous shaking. The overnight culture is transferred at a 1% inoculum into 50 mL of LB liquid medium containing 20 μg/mL of tetracycline and cultured at 37° C. with vigorous shaking until the OD_{600 nm} reaches approximately 0.8. Xylose with a final concentration of 0.5% is added, and the culture is continued at 37° C. for 30 hours (h), the culture is then centrifuged at 12,000 revolutions per minute (rpm) for 10 min to collect the supernatant. The target protein in the fermentation supernatant is purified using a Ni²⁺ affinity chromatography column, eluted with 200 millimoles per liter (mmol/L) of imidazole buffer, and the purified mutant is obtained. The purity of the mutant is detected by SDS-PAGE, and the concentration is determined by the Bradford method.

Screening of mutants: the specific enzyme activity of type III pullulan hydrolase TK-PUL and its mutants is measured using cassava starch as the substrate. 10 microliters (μL) of enzyme solution is mixed with 490 μL of 50 mmol/L2-(N-morpholine) ethanesulfonic acid (MES) containing 1% (m/v) of cassava starch, and the pH is 4.5, after reacting at 100° C. for 30 min, the reaction is immediately stopped by placing the mixture in an ice bath, and the reducing sugar content in the reaction system is determined using the 3,5-dinitrosalicylic acid method. The reducing sugar produced is converted to maltose equivalents using a maltose standard working curve. One unit of enzyme activity (U) is defined as the amount of enzyme that catalyzes the production of 1 micromole (μmol) of maltose per minute under the specified reaction conditions.

The specific enzyme activity of type III pullulan hydrolase TK-PUL on cassava starch is 48.68±1.32 Unit per milligram (U/mg). The specific enzyme activities of the mutants on cassava starch are shown in Table 2. The results show that the specific enzyme activities of mutants C282K, C282R, C283D, C283E, and C283S are significantly higher than that of type III pullulan hydrolase TK-PUL. Specifically, the specific enzyme activity of mutant C282K is 49.35 U/mg, a 1.01-fold increase; C282R is 49.92 U/mg, a 1.03-fold increase; C283D is 65.08 U/mg, a 1.34-fold increase; C283E is 68.93 U/mg, a 1.42-fold increase; and C283S is 59.90 U/mg, a 1.23-fold increase.

TABLE 2
specific enzyme activities of mutants on cassava starch

| | Specific Enzyme | | Specific Enzyme |
|---|---|---|---|
| | Activity | | Activity |
| Mutant | (U/mg | Mutant | (U/mg) |
| C282A | 38.94 ± 2.02 | C283A | 35.61 ± 2.15 |
| C282G | 37.98 ± 2.53 | C283G | 34.19 ± 2.03 |
| C282V | 31.08 ± 1.65 | C283V | 30.21 ± 1.93 |
| C282L | 26.99 ± 1.53 | C283L | 23.98 ± 1.25 |
| C282I | 28.93 ± 1.53 | C283I | 19.69 ± 1.03 |
| C282P | 30.61 ± 2.02 | C283P | 26.76 ± 1.54 |
| C282F | 33.08 ± 1.81 | C283F | 28.97 ± 1.67 |
| C282W | 32.11 ± 1.93 | C283W | 28.06 ± 1.57 |
| C282D | 40.18 ± 2.19 | C283D | 65.08 ± 2.82 |
| C282E | 41.06 ± 1.78 | C283E | 68.93 ± 2.91 |
| C282T | 39.91 ± 1.89 | C283T | 46.16 ± 2.43 |
| C282N | 37.57 ± 1.87 | C283N | 48.06 ± 2.61 |
| C282K | 49.35 ± 2.51 | C283K | 45.27 ± 2.51 |
| C282S | 45.75 ± 2.54 | C283S | 59.90 ± 2.97 |
| C282R | 49.92 ± 1.93 | C283R | 43.62 ± 2.19 |
| C282H | 46.08 ± 2.30 | C283H | 44.19 ± 2.57 |
| C282Q | 45.39 ± 2.70 | C283Q | 46.18 ± 3.01 |
| C282Y | 40.09 ± 2.62 | C283Y | 43.05 ± 2.17 |
| C282M | 39.17 ± 2.19 | C283M | 40.37 ± 3.02 |

### (3) Construction and Screening of Combinatorial Mutants

Construction of combinatorial mutants: the beneficial mutations that improve the specific enzyme activity of type III pullulan hydrolase are combined to construct combinatorial mutants C282K/C283D, C282K/C283E, C282K/C283S, C282R/C283D, C282R/C283E, and C282R/C283S. The construction method is illustrated using the combinatorial mutant C282K/C283D as an example.

Using the recombinant plasmid pSTOP1622-tkpulC282K as a template and primers C282K/C283D-F and C282K/C283D-R from Table 1, PCR amplification is performed. The PCR amplification conditions are as follows: 94° C. for 5 min; 94° C. for 30 s, 55° C. for 20 s, 68° C. for 4 min, 35 cycles; and 68° C. for 10 min. The amplified product is treated with Dpn I enzyme and then electroporated into E. coli JM109 competent cells, coated with LB plate containing 100 g/mL of ampicillin and cultured overnight at 37° C. Single transformants are picked from the LB plate, and the recombinant plasmid pSTOP1622-tkpulC282K/C283D is extracted. Using this plasmid as a template, PCR amplification is performed with primers P1 and P2, and the amplified DNA is sent to Sangon Biotech Co., Ltd. for sequencing to confirm the presence of the tkpulC282K/C283D mutation. The correctly sequenced recombinant plasmid pSTOP1622-tkpulC282K/C283D is transformed into B. subtilis WB600 to obtain the engineered strain B. subtilis WB600/pSTOP1622-tkpulC282K/C283D.

The construction of other combinatorial mutants follows the same method as for combinatorial mutant C282K/C283D.

Induced expression and purification of combinatorial mutants: the engineered strain is inoculated into 20 mL of LB liquid medium containing 20 μg/mL of tetracycline and cultured overnight at 37° C. with vigorous shaking. The overnight culture is transferred at a 1% inoculum into 50 mL of LB liquid medium containing 20 μg/mL of tetracycline and cultured at 37° C. with vigorous shaking until the OD_{600 nm} reaches approximately 0.8. Xylose with a final concentration of 0.5% is added, and the culture is continued at 37° C. for 30 h, the culture is then centrifuged at 12,000 rpm for 10 min to collect the supernatant. The target protein in the fermentation supernatant is purified using a Ni²⁺ affinity chromatography column, and eluted with 200 mmol/L of imidazole buffer, and then the purified mutant is obtained. The purity of the mutant is detected by SDS-PAGE, and the concentration is determined by the Bradford method.

Screening of mutants: the specific enzyme activity of type III pullulan hydrolase TK-PUL and its mutants is measured using cassava starch as the substrate. 10 μL of enzyme solution is mixed with 490 μL of 50 mmol/L2-(N-morpholine) ethanesulfonic acid (MES) containing 1% (m/v) cassava starch, and the pH is 4.5, after reacting at 100° C. for 30 min, the reaction is immediately stopped by placing the mixture in an ice bath, and the reducing sugar content in the reaction system is determined using the 3,5-dinitrosalicylic acid method. The reducing sugar produced is converted to maltose equivalents using a maltose standard working curve. One unit of enzyme activity (U) is defined as the amount of enzyme that catalyzes the production of 1 μmol of maltose per minute under the specified reaction conditions.

The specific enzyme activities of combinatorial mutants on cassava starch are shown in Table 3. The results show that the specific enzyme activities of combinatorial mutants are significantly higher than that of type III pullulan hydrolase TK-PUL. Specifically, the combinatorial mutant C282R/C283E has the highest specific enzyme activity at 89.08 U/mg, a 1.83-fold increase.

TABLE 3
specific enzyme activities of combinatorial
mutants on cassava starch

| Combinatorial | Specific Enzyme |
|---|---|
| Mutant | Activity (U/mg) |
| C282K/C283D | 65.97 ± 3.03 |
| C282K/C283E | 67.08 ± 3.56 |
| C282K/C283S | 59.98 ± 3.21 |
| C282R/C283D | 80.63 ± 3.39 |
| C282R/C283E | 89.08 ± 3.43 |
| C282R/C283S | 60.02 ± 2.94 |

### Embodiment 2: Enzymatic Properties of Type III Pullulan Hydrolase Mutant C282R/C283E

### (1) Optimal Reaction Temperature of Type III Pullulan Hydrolase Mutant C282R/C283E

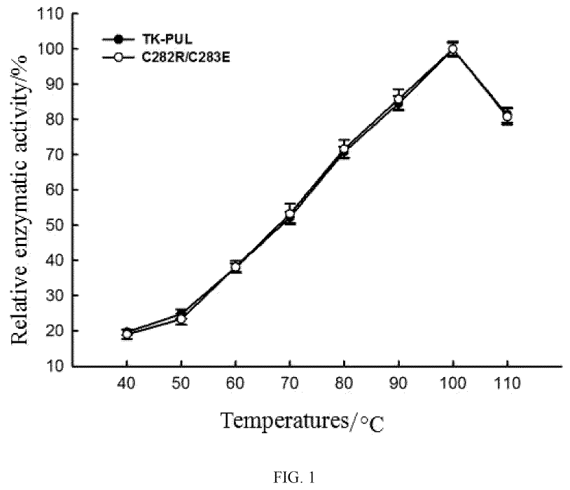

10 μL of enzyme solution is mixed with 490 μL of 1% (m/v) cassava starch in 50 mmol/L MES buffer (pH 4.5), and the mixture is reacted at temperatures ranging from 40° C. to 110° C. for 30 min, and the specific enzyme activity is measured at different temperatures. The highest specific enzyme activity is defined as 100%, and the relative enzyme activity is plotted against temperature to determine the optimal reaction temperature. The optimal reaction temperatures of type III pullulan hydrolase TK-PUL and mutant C282R/C283E are shown in FIG. 1. Both type III pullulan hydrolase TK-PUL and mutant C282R/C283E have an optimal reaction temperature of 100° C.

### (2) Optimal Reaction pH of Type III Pullulan Hydrolase Mutant C282R/C283E

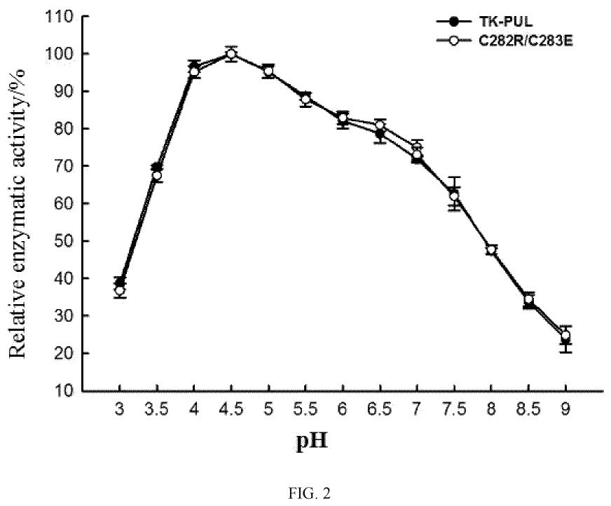

10 μL of enzyme solution is mixed with 1% (m/v) cassava starch solutions at different pH values and reacted at 100° C. for 30 min, and the specific enzyme activity is measured. The highest specific enzyme activity is defined as 100%, and the relative enzyme activity is plotted against pH to determine the optimal reaction pH. Different buffers are used to prepare 1% (m/v) cassava starch solutions at different pH values: 50 mmol/L of MES (pH 3.0-7.0) and 50 mmol/L of 3-(N-Morpholino)propanesulfonic acid (MOPS) (pH 7.0-9.0). The optimal reaction pH values of type III pullulan hydrolase TK-PUL and mutant C282R/C283E are shown in FIG. 2. Both type III pullulan hydrolase TK-PUL and mutant C282R/C283E have an optimal reaction pH of approximately 4.5, and the pH-activity curves of TK-PUL and mutant C282R/C283E are similar.

### (3) Thermal Stability of Type III Pullulan Hydrolase Mutant C282R/C283E at 90° C.

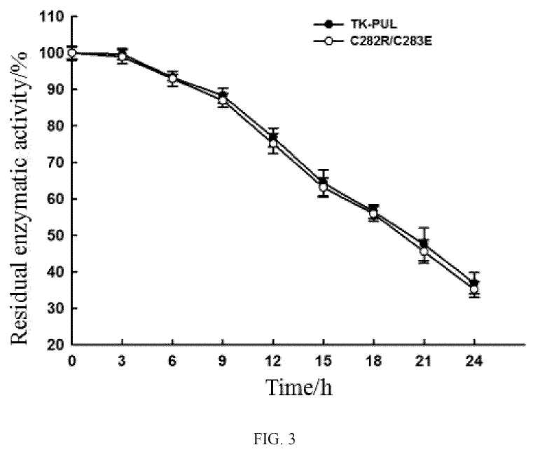

The enzyme solution is incubated at 90° C., and samples are taken by time gradient. The specific enzyme activity is measured using 1% (m/v) cassava starch in 50 mmol/L of MES buffer (pH 4.5) as the substrate at 100° C. for 30 min. The enzyme activity of the untreated enzyme solution is defined as 100%, and the relative enzyme activity is plotted against time to evaluate the thermal stability of the enzyme. The thermal stability of type III pullulan hydrolase TK-PUL and mutant C282R/C283E at 90° C. is shown in FIG. 3. Both type III pullulan hydrolase TK-PUL and mutant C282R/C283E have a half-life of approximately 20 hours at 90° C.

The above enzymatic property measurements show that the optimal reaction temperature, optimal reaction pH, and thermal stability of mutant C282R/C283E are consistent with those of TK-PUL.

### Embodiment 3: Preparation of Linear Maltooligosaccharides by Type III Pullulan Hydrolase Mutant C282R/C283E

Method for preparing linear maltooligosaccharides using type III pullulan hydrolase and product analysis: 100 mL of 30% (m/v) cassava starch slurry (50 mmol/L of MES, pH 4.5) is prepared and stirred at 400 revolutions per minute (r/min) in a 70° C. water bath for 10 min. Then, type III pullulan hydrolase is added to the cassava starch slurry at enzyme dosages of 1 milligram (mg), 2 mg, and 4 mg per gram of dry cassava starch, respectively. The mixtures are treated at 100° C. with the same stirring frequency for 10 min, followed by incubation at 90° C. with the same stirring frequency for 6 h, 12 h, 18 h, 24 h, or 36 h, and then the reaction is stopped. After the reaction, 1 mL of the reaction mixture is taken, placed in an ice bath, and centrifuged at 12,000×g for 10 min to collect the supernatant. Half of the supernatant is freeze-dried to obtain dry starch sugar, which is weighed. The other half of the supernatant is diluted 10,000 times and filtered through a 0.22 μm aqueous filter membrane to obtain the product for analysis. Using linear maltooligosaccharides G2-G10 as standards, the content of linear maltooligosaccharides G2-G10 components in the product is determined by High-Performance Anion-Exchange Chromatography with Pulsed Amperometric Detection (HPAEC-PAD), and the substrate conversion rate of linear maltooligosaccharides G2-G10 is calculated. The substrate conversion rate of linear maltooligosaccharides G2-G10 components is calculated as follows: G2-G10 substrate conversion rate=[(G2−G10 component content×dry starch sugar mass g×200)/30 g (dry cassava starch mass)]×100%.

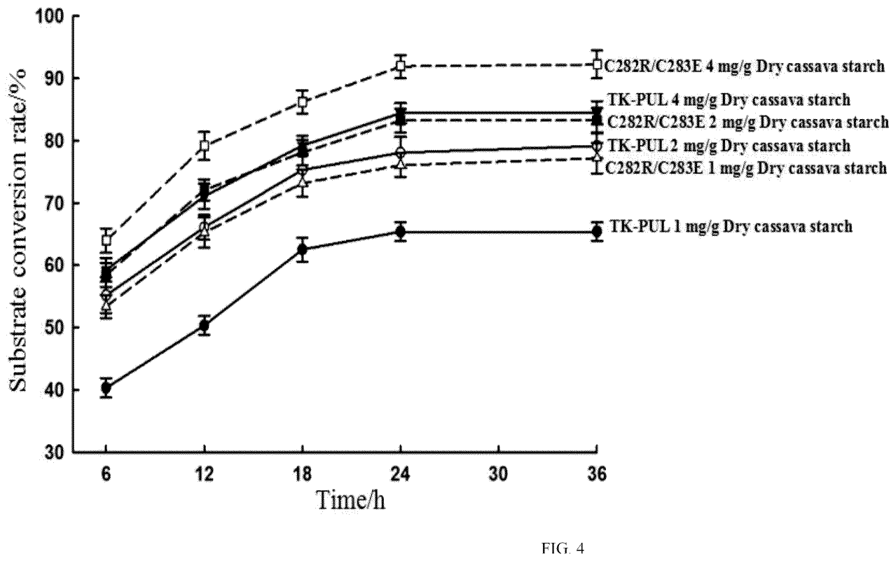

The substrate conversion rates of G2-G10 components in the product prepared from cassava starch by type III pullulan hydrolase TK-PUL and mutant C282R/C283E are shown in FIG. 4. As shown in FIG. 4, the substrate conversion rate of G2-G10 components increases with reaction time until it is stabilized. The substrate conversion rate also increases with the increase in the amount of type III pullulan hydrolase added. When the dosage of type III pullulan hydrolase is 1 mg enzyme per gram of dry cassava starch and the reaction time is 36 h, the substrate conversion rate for TK-PUL is 65.41%, while that for mutant C282R/C283E is 77.19%. When the dosage of type III pullulan hydrolase is 2 mg enzyme/g of dry cassava starch and the reaction time is 36 h, the substrate conversion rate for TK-PUL is 79.15%, while that for mutant C282R/C283E is 83.29%. When the dosage of type III pullulan hydrolase is 4 mg enzyme/g dry of cassava starch and the reaction time is 36 h, the substrate conversion rate for TK-PUL is 84.50%, while that for mutant C282R/C283E is 92.23%. Under the same conditions, the substrate conversion rate of mutant C282R/C283E is higher than that of TK-PUL. Compared with type III pullulan hydrolase TK-PUL, type III pullulan hydrolase mutant C282R/C283E has greater potential for application in the production of linear maltooligosaccharides.

The above-described embodiments are only optional implementations of the disclosure and are not intended to limit the scope of the disclosure. Without departing from the spirit of the disclosure, various modifications and improvements made by those skilled in the art to the technical schemes of the invention shall fall within the scope of protection defined by the claims of the disclosure.

What is claimed is:

1. A mutant of Type III pullulan hydrolase, wherein an amino acid sequence is shown in SEQ ID NO. 3.

2. An encoding gene encoding the mutant of Type III pullulan hydrolase according to claim 1.

3. The encoding gene according to claim 2, wherein a nucleotide sequence is shown in SEQ ID NO. 4.
